(19) United States
(12) Patent Application Publication
(10) Pub. No.: US 2005/0250849 A1
(43) Pub. Date: Nov. 10, 2005

(54) Basic amino acid - methionine hydroxy analog compositions

(75) Inventors: Steve Lorbert, St. Louis, MO (US); Charles S. Schasteen, St. Louis, MO (US); Farooq Uraizee, Eureka, MO (US)

Correspondence Address: PALMER & DODGE, LLP;KATHLEEN M. WILLIAMS, 111 HUNTINGTON AVENUE, BOSTON, MA, 02199, US

(73) Assignee: Novus International, Inc.

(21) Appl. No.: 11/119,252

(22) Filed: Apr. 29, 2005

Related U.S. Application Data
(60) Provisional application No. 60/566,722, filed on Apr. 30, 2004.

(51) Int. Cl. A61K 31/205

(52) U.S. Cl. 514/554; 562/562

(57) ABSTRACT

Simple, cost-effective and convenient compositions comprising basic amino acids and 2-hydroxy-4-methylthiobutanoic acid (HMBA) are disclosed. The compositions have many uses, including as food supplements for animal and human food.

### RELATED APPLICATIONS

This application claims priority to the U.S. Provisional Application Ser. No. 60/566,722, filed on Apr. 30, 2004.

### FIELD OF INVENTION

This invention relates to blends of basic amino acids with 2-hydroxy-4-methylthiobutanoic acid.

### BACKGROUND

Amino acids, in particular essential amino acids such as leucine, isoleucine, valine, methionine, threonine, lysine, histidine, phenylalanine, and tryptophan, are essential for humans and animals. Therefore, to obtain ideal growth, amino acids must be provided in the diet of humans and animals. For this reason, amino acids are typically supplemented within foods and animal feed. Aqueous compositions comprising amino acids are particularly useful in supplementing animal feed. Such compositions mix readily and thoroughly in the feed mixer and are absorbed by the feed particles, becoming inseparable from the feed mix. In contrast, powder sources of amino acids tend to separate from the feed over time. Powder sources of nutrient supplements can account for up to one third of airborne dust in a typical poultry feed mill. Any puncturing of bags or excess movement of the product can cause product loss and subsequently dust. In contrast, a liquid system eliminates the airborne dust attributed to powder forms of supplement, resulting in a cleaner, safer mill environment. Liquid amino acid addition systems are often automated and therefore require less labor for their addition, as compared with bagged ingredients which must be handled manually. This results in a more cost-effective means for adding amino acids to food and feed.

U.S. Pat. No. 4,617,155, which discloses lysine salt crystals comprising an equimolar amount of lysine and the methionine analog, 2-hydroxy-4-methylthiobutyric acid, states that “[t]he production of lysine-2-hydroxy-4-methylthiobutyric acid equimolar salt crystals is effected by causing lysine and 2-hydroxy-4-methylthiobutyric acid to react with each other in an aqueous medium and subsequently concentrating, crystallizing, and separating the resulting reaction mixture.” However, crystal salts require milling and mixing. It is easier to mix into feed in the liquid state.

In addition to the general need for food supplements for both animals and humans, a particular need exists for a nitrogen-free nutritional supplement for individuals with or at risk of developing renal insufficiency. Patients with chronic renal insufficiency, kidney disease, or other similar disorders suffer from the effects of the build up of toxic chemicals in the blood, for example, nitrogenous compounds such as ammonia, increased levels of urea, creatinine and other toxic products and metabolites. Hemodialysis is typically the treatment of end stage forms of chronic renal insufficiency.

Thus, a need exists for a form of a human nutritional supplement or an animal feed supplement that is easy and cost-effective to manufacture, and which avoids the problems associated with powdered forms of supplements. A need also exists for an alternative dietary supplement for patients with chronic renal insufficiency, such as kidney failure, which delays the need for dialysis treatment by reducing levels of nitrogen in the blood. Further, a need exists for an improved dialysate composition that can effectively reduce calcium from the blood of a patient undergoing hemodialysis.

Attempts at prophylactic treatment of renal failure have focused on altering the patient's diet. A major goal of such treatment is to minimize the burden on the kidneys. For example, it has been shown that prognosis of chronic renal failure patients is vastly improved by providing a very low protein diet in order to minimize build up of urea in the blood, and can delay dialysis treatment for up to one year (Walser (1994) Curr Opin Nephrol Hypertens. 3, 301-4). Patients were prescribed a very low protein diet (typically around 0.3 g/kg) and supplemented by 10 to 20 g/d essential amino acids or ketoacids. The use of alpha-ketoacids, or deaminated amino acids, function by recycling the circulating nitrogen to create proteins, further reducing the buildup of toxic levels of nitrogen in the blood (see, for example, U.S. Pat. No. 4,352,814). However, many ketoacids are expensive and difficult to prepare.

Conventional hemodialysis is associated with profound disturbances in calcium and phosphate metabolism. Hyperphosphataemia (excessive levels of phosphate in the blood) affects as many as 80% of patients undergoing dialysis for end-stage renal disease. Recent studies suggest that the calcium-based phosphate binders taken by patients with end-stage renal disease on hemodialysis may contribute to the progression of cardiac calcification, an indicator of cardiovascular disease. Cardiovascular disease accounts for nearly 50% of all deaths in dialysis patients (Davies & Hruska, (2001) Kidney Int. 60, 472-9).

### SUMMARY OF THE INVENTION

The invention relates to compositions comprising a basic amino acid and 2-hydroxy-4-methylthiobutanoic acid, and methods for supplementing food with such compositions.

In one aspect, a composition comprising a basic amino acid and 2-hydroxy-4-methylthiobutanoic acid is provided. The mass ratio of basic amino acid to 2-hydroxy-4-methylthiobutanoic acid is between 4:1 and 1:4.

In another aspect, a nutritional supplement comprising a blend of a basic amino acid and 2-hydroxy-4-methylthiobutanoic acid is provided. The mass ratio of basic amino acid to 2-hydroxy-4-methylthiobutanoic acid is between 4:1 and 1:4.

In yet another aspect, a method of supplementing food is provided, which comprises the following steps: preparing a mixture of a basic amino acid and 2-hydroxy-4-methylthiobutanoic acid, wherein the mass ratio of basic amino acid to 2-hydroxy-4-methylthiobutanoic acid is between 4:1 and 1:4; and, combining the mixture with food.

### BRIEF DESCRIPTION OF THE FIGURES

FIG. 1 is a diagrammatic view of a portable storage and dispensing system from U.S. Pat. No. 5,533,648.

### DETAILED DESCRIPTION

The present invention is based upon the discovery that stable aqueous preparations of basic amino acids and 2-hydroxy-4-methylthiobutanoic acid (HMBA) can be produced by combining basic amino acids with 2-hydroxy-4-methylthiobutanoic acid. The preparations described herein are useful for supplementing food or can be used as a nutritional supplement.

The term “basic amino acid”, as used herein, refers to an amino acid which is positively charged at pH 7. Examples of basic amino acids, include, but are not limited to, lysine, arginine, and histidine and the salts thereof (such as lysine hydrochloride or lysine sulfate). A “basic amino acid” can be in any liquid or dry forms known in the art, and it can be a purified amino acid or the salt thereof (i.e., at least 95% by weight), or it can contain less than 95% by weight of amino acid or the salt thereof, but also contain other components (e.g., culture broth, and/or whole bacteria cells) in addition to the amino acid. In one embodiment, the basic amino acid is in liquid form. Many preparations of basic amino acids are commercially available, for example, ADM Liquid L-Lysine™, Feed Grade (50% by weight, Archer Daniels Midland Company, Decatur, Ill.), or Liquid Lysine 60™, Feed Grade (60% by weight, Ajinomoto Heartland LLC, Chicago, Ill.), the latter of which has a chloride content of about 2.5 to 3%. In one embodiment, the basic amino acid is lysine or a salt thereof.

The usual commercial form of 2-hydroxy-4-methylthiobutanoic acid (HMBA) is the optically racemic D,L-2-hydroxy-4-methylthiobutanoic acid mixture. It should be understood that while the 2-hydroxy-4-methylthiobutanoic acid compositions referred to hereinafter are racemic mixtures, the individual D- and L-isomers of 2-hydroxy-4-methylthiobutanoic acid can be converted to the 2-hydroxy-4-methylthiobutanoic acid compositions by procedures known in the art. Hence, as used herein, the terms “HMBA” and “2-hydroxy-4-methylthiobutanoic acid” refer either to the D- or L-isomer of 2-hydroxy-4-methylthiobutanoic acid or any mixture of the two above-described isomers thereof. 2-hydroxy-4-methylthiobutanoic acid is available in liquid form (typically 88% minimum by weight) from several commercial sources, including Alimet™ (Novus International, St. Louis, Mo.) and Rhodimet™ AT 88 (Adisseo, Antony, France).

The compositions described herein are produced by mixing a basic amino acid with 2-hydroxy-4-methylthiobutanoic acid at a mass ratio of basic amino acid to 2-hydroxy-4-methylthiobutanoic acid between 4:1 and 1:4. The mass ratio of basic amino acid to 2-hydroxy-4-methylthiobutanoic acid can between 3:1 to 1:1.5, for example between 2.5:1 and 1:1.25, between 2:1 and 1:1, or between 1.5:1 and 1:1.

When the total concentration of basic amino acid and 2-hydroxy-4-methylthiobutanoic acid exceed 77% by weight, the compositions have a tendency to form solids. At lower concentrations, the compositions described herein remain aqueous at low ambient temperatures, and thus can be delivered conveniently to a commercial setting where they can, for example, be combined with food or animal feed. In order to maintain an aqueous state, the total concentration of basic amino acid and 2-hydroxy-4-methylthiobutanoic acid in the composition is maintained at less than 77% by weight, for example less than 70% by weight, or less than 65% by weight.

As used herein, “food” refers to a substance which is ingested by humans or other animals. As used herein, “food” refers to any solid or liquid substance, raw or processed, which is ingested for nutritional purposes, in that it provides a source of metabolizable energy, supplementary or necessary vitamins or co-factors, roughage or otherwise beneficial effect upon ingestion by an animal. In certain embodiments, “food” refers to animal feed.

As used herein, the term “animal feed” refers to feed for animals, i.e., feed that can be used in the animal husbandry field and is suitable to be fed to, for example, meat-producing animals to supply part or all of the meat-producing animal's nutrient requirements. The feed is typically a mixture or grains or grain derived components and one or more nutritional supplements, e.g., amino acids, fat, vitamins, minerals, and the like.

Also disclosed herein is a method of supplementing food, comprising the steps of preparing an aqueous mixture of a basic amino acid and 2-hydroxy-4-methylthiobutanoic acid, wherein the mass ratio of basic amino acid to 2-hydroxy-4-methylthiobutanoic acid is between 4:1 and 1:4; and, combining the mixture with the food. In one embodiment, the basic amino acid and 2-hydroxy-4-methylthiobutanoic acid used in this method are in liquid form.

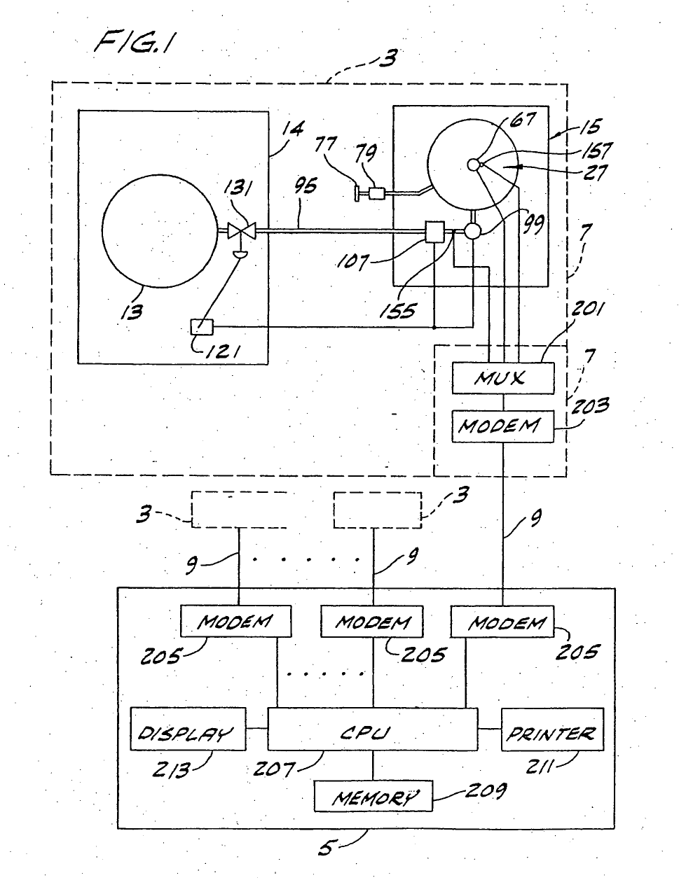

The aqueous mixture can be prepared at a central manufacturing facility prior to delivery to a customer. Alternatively, the aqueous mixture can be conveniently prepared directly at the customer's site by using a portable storage and dispensing systems using components similar to those disclosed, for example, in U.S. Pat. No. 5,533,648, which is herein incorporated by reference in its entirety. In one embodiment of such a mixing device, the system consists of one tank containing liquid HMBA and another tank containing a liquid basic amino acid, each tank associated with a level-measuring device. For purposes of this discussion, numeric reference is made to the components of a mixing device useful for the methods described herein as identified in FIG. 1 hereof, which corresponds to FIG. 1 of U.S. Pat. No. 5,533,648. The tanks (item 27 of FIG. 1), designed for holding a liquid chemical, are generally cylindrical in shape, and are contained within a common dike and enclosure. The tanks are of a suitable material, for example a translucent plastic (e.g., high-density, cross-linked polyethylene) enabling the level of liquid in the tank to be seen from outside the tank. The capacity of the tanks will vary according to need (1000 gallons is common). The measuring device (item 67 of FIG. 1) projects down from the top of the tank for measuring the level of liquid in the tank. This device can be a level sensor, such as an ultrasonic sensor of the type sold under the trademark “Level Devil” by Electronic Sensors, Inc. of Wichita, Kans., and is operable for sending a signal indicative of the level of liquid in the tank to the central station so that the current amount of liquid in the tank can be closely monitored. In one embodiment, the measuring device includes a digital readout mounted at a convenient height on a sidewall of the upper part of the enclosure so that it is readily visible by a person looking through the open closure. The system further comprises individual communications lines from each tank to a common multiplexer (item 201 of FIG. 1) and modem (item 203 of FIG. 1). Output from output from the HMBA level-measuring device can be multiplexed with the output from the basic amino acid level-measuring device by the multiplexer 201. The multiplexed output signal from the multiplexer is provided to a modem 203 which communicates with a modem (item 205 of FIG. 1) at the central station. A line interconnecting modems 203 and 205 can be a dedicated telephone line. Alternatively, modem 203 can be an auto-dial system which automatically interconnects with modem 205 at preprogrammed times via a telephone line to download the multiplexed signals. Although the multiplexer 201 is illustrated as a separate element, it is contemplated that it can be part of the measuring device 67, in which case the sensors 155 and 157 would be connected to the measuring device 67 which in turn would be connected to the modem 203.

Using such a configuration, liquid HMBA and liquid basic amino acids could be added simultaneously in the proper proportions using separate pumps (for example, item 99) and metering devices (107) which supply a common supply line (95) and automated valve (131). Alternatively, HMBA and the basic amino acid solution could be added sequentially using a common Pump (99) and Metering Device (107) which supply a common supply line and automated valve.

When used as a nutritional supplement, it is desirable that the compositions have physical properties which are compatible with systems designed for mixing with food. The compositions include aqueous compositions which are stable at ambient temperatures, which remain liquid at temperatures between 0° C. and −16° C., and are therefore possess favorable storage properties. In one embodiment, the preparations include aqueous compositions with acceptable viscosity as to permit the use of conventional pumps in mixing with animal feed. To permit efficient mixing and delivery using conventional pumps, the viscosity of the preparations is less than 10,000 centi-Stokes at temperatures between 4° C. and 30° C., for example less than 5,000 centi-Stokes, or than 2,500 centi-Stokes.

The preparations described herein are blends of an acid (HMBA) and a base (a basic amino acid). The ratios of the acid and base, therefore, will influence the pH of the resulting blend. To minimize corrosion of metal piping systems used in the delivery of the mixtures, it is beneficial for the compositions to possess pH values which are close to neutrality, for example, between pH 4 and pH 11, or between pH 6 and pH 10.

These compositions can also be incorporated as part of a nutritional supplement. As used herein, the terms “nutrient supplement” and “nutritional supplement” refer to a composition that is intended to supplement the diet in humans or other animals. A nutritional supplement or nutrient supplement includes any dietary substance used in animals to supplement the diet by increasing total dietary intake; or a concentrate, metabolite, constituent, extract, etc. A nutrient supplement or nutritional supplement includes any product that is intended for ingestion in tablet, capsule, powder, soft-gel, gel-cap, or liquid form.

When in the form of a nutritional supplement, the compositions described herein can be in a form for separate administration, such as a capsule, a tablet, a powder, a sachet, a liquid composition (e.g. droplets) or a similar form, and in one embodiment containing a unit dose of amino acid blends. Such a supplement can further comprise one or more adjuvants, carriers or excipients suitable for use in food supplements, as well as one or more of the further components and/or additives described below.

The nutritional supplement can also be in the form of a powder, a liquid composition (e.g., droplets or elixirs) or a similar form, which can be added to or mixed with a suitable food (composition) or a suitable liquid or solid carrier, for the preparation of a food or drink which is ready for consumption. For instance, the nutritional supplement can be in the form of a powder which can be mixed with, and/or reconstituted with, water, milk, fruit juice, infant drinks, oral rehydration solution, etc. It can also be in the form of a powder or liquid that can be mixed with solid foods or with foods with a high-water-content, such as fermented foods, for example yogurt.

The nutritional compositions can also be in the form of a solid, semi-solid or liquid food which is ready for consumption. Such a food can comprise, in addition to the compositions of the present invention, a food or food base known per se, and can, for instance, be prepared by adding a nutritional supplement as described above to a food or food base; adding the compositions to a food or food base; and/or, incorporating the compositions to a food or food base during the preparation thereof.

The nutritional compositions of the invention, whether in the form of a food for consumption or a food supplement, can further comprise all desired components and/or additives for use in foods or food supplements, including but not limited to flavors, colorings, preservatives, sugar, etc. The composition can contain one or more peptides and/or proteins, lipids, carbohydrates, vitamins, minerals and trace elements, in usual amounts.

The compositions described herein are particularly beneficial as dietary supplements for patients with kidney disorders, for example chronic renal failure. Thus, in one embodiment, the nutritional supplement of the present invention comprises a composition comprising a basic amino acid and 2-hydroxy-4-methylthiobutanoic acid, wherein the mass ratio of basic amino acid to 2-hydroxy-4-methylthiobutanoic acid is between 4:1 and 1:4. The supplement can further comprise essential amino acids, alpha-ketoacids, or a mixture thereof. As used herein, the term “alpha-ketoacids” refers generally to nitrogen-free analogs of amino acids, typically deaminated alpha-keto analogs of essential amino acids, for example, α-ketoleucine (α-ketoisocaproate), α-ketoisoleucine, α-ketophenylalanine, and α-ketovaline. In one embodiment, the nutritional supplement further comprises a mixture of essential amino acids, alpha-ketoacid and hydroxy acid analogs of amino acids. In another embodiment, the nutritional supplement provides the daily requirement for each essential amino acid (Lehninger, Principles of Biochemistry, Worth Publishers, New York, 1982).

As evidenced by the reduced skeletal muscle degradation upon supplementation of humans and animals with 2-hydroxy-4-methylthiobutanoic acid (Stewart et al., (1982) Muscle & Nerve, 5, 197-201; Dibner and Knight (1986) Poult. Sci. 65, Suppl. 1, 34; Phillips et al., (2003) J. Dairy Sci. 86, 3634-3647), the compositions described herein are particularly beneficial as nutritional supplements to reduce muscle protein degradation in animals and humans. Supplementation of diet with the compositions described herein can be useful for the prevention of the degradation of muscle in humans suffering from malnutrition, uremia, liver disease, cancer, diabetes, sepsis, as well as from surgery or other physical trauma. In addition, the nutritional supplements can be useful as feed supplements for fowl or ruminant animals to minimize nitrogen wasting.

HMBA is known to form tight complexes with divalent cations, in particular, with calcium. The compositions described herein, which comprise a basic amino acid and 2-hydroxy-4-methylthiobutanoic acid, are therefore also useful as a composition for calcium removal or exchange. Therefore, in another aspect, dialysate precursor compositions are provided, comprising a basic amino acid and 2-hydroxy-4-methylthiobutanoic acid, wherein the mass ratio of basic amino acid to 2-hydroxy-4-methylthiobutanoic acid is between 4:1 and 1:4, a buffer, water; chloride, and at least one physiologically-acceptable cation. Upon dilution with water, the precursor composition described herein forms a composition useful for either hemodialysis or peritoneal dialysis. As used herein, the term “chloride” refers to anionic chloride and includes anionic chloride and the salts thereof, for example from physiological cations. Examples of physiologically acceptable cations include, but are not limited to, hydrogen (proton), basic amino acids, metal cations, and ammonium cations. In one embodiment, the composition containing chloride salts contains a mixture of physiological acceptable cations.

As used herein, the term “buffer” refers to a compound that is known to be safe for use in dialysis formulations and that has the effect of controlling the pH of the formulation at the pH desired for the formulation. Buffers for controlling pH at a moderately acid pH to moderately basic pH include, for example, such compounds as phosphate, acetate, citrate, lactate, arginine, histidine and lysine.

As used herein, the terms “treated water” and “MMI-quality water” refer to water that has been treated to meet the purity requirements established by the Association for the Advancement of Medical Instrumentation (MMI) for dialysate compositions, and which is free from pyrogens, and live bacteria or other microorganisms. A monograph describing water treatment for dialysate, monitoring of water treatment systems, and regulation of water treatment systems is available from MMI (Water Quality for Dialysis, in Standards Collection, Vol. 3, Dialysis, Section 3.2, 1998, (3 ed.) AAMI, Arlington, Va.). In one embodiment, the components of the dialysate composition are at least United States Pharmacopoeia (USP)-grade purity, which is generally a purity of about 95%. In one embodiment, the purity of the components is at least about 95%, for example at least about 98%, or at least about 99%.

In one embodiment, the precursor composition comprises a composition comprising a basic amino acid and 2-hydroxy-4-methylthiobutanoic acid. In another embodiment, the mass ratio of basic amino acid to 2-hydroxy-4-methylthiobutanoic acid is between 4:1 and 1:4. In still another embodiment, the 2-hydroxy-4-methylthiobutanoic acid ranges from about 20 to about 900 milli-equivalents per liter (mEq/L), chloride ranges from about 1,000 to about 7,000 mEq/L, and a physiologically acceptable cation ranges from about 20 to about 900 mEq/L. In another embodiment, the physiologically acceptable cation is lysine. As used herein, “mEq/L” refers to the concentration of a particular component (solute), more specifically the number of milliequivalents of solute per liter of water. mEq/L is calculated by multiplying the concentration of the solute (in millimoles per liter) by the number of charged species (groups) per molecule of solute.

In another aspect, a buffered dialysate composition is provided, which comprises water, chloride, a composition comprising a basic amino acid and 2-hydroxy-4-methylthiobutanoic acid, wherein the mass ratio of basic amino acid to 2-hydroxy-4-methylthiobutanoic acid is between 4:1 and 1:4, a buffer, a base including bicarbonate, and one physiologically acceptable cation.

In one embodiment, the dialysate composition described herein includes one or more sugars selected from the group consisting of glucose, poly(glucose) and fructose at a concentration of less than about 45 grams per liter (g/L). In another embodiment, the dialysate composition has a pH of between about 5 to about 9 at a temperature between 25° C. to 40° C., and typically has a pH of between 6.4 and 8.2, or between 7.2 and 7.4.

In one preferred embodiment, the dialysate composition comprises chloride at a concentration ranging from about 20 to about 200 mEq/L, 2-hydroxy-4-methylthiobutanoic acid at a concentration ranging from about 0.1 to about 60 mEq/L; a buffer; a base such as bicarbonate at a concentration from about 20 to about 50 mEq/L; and at least one physiologically acceptable cation at a concentration ranging from about 0.1 to about 60 mEq/L. In yet another embodiment, the physiologically acceptable cation is lysine.

The following examples demonstrate experiments performed and contemplated by the present inventors in making and carrying out the invention. It is believed that these examples include a disclosure of techniques which serve to both apprise the art of the practice of the invention and to demonstrate its usefulness. Those of skill in the art will appreciate that the techniques and embodiments disclosed herein are preferred and non-limiting embodiments only, and that in general numerous equivalent methods and techniques may be employed to achieve the same result.

### EXAMPLES

In the following examples, amino acids from various sources were mixed with a commercial preparation of HMBA (Alimet®, Novus International, St. Louis, Mo.) in different quantities to give the desired lysine to HMBA ratio in the blend. The viscosity of these blends were analyzed using Cannon-Fenske viscometers temperatures of 25° C. and 4° C. and is reported in cSt (centi-Stokes). The density of the blends was also evaluated. The physical state of the blends was also determined after cooling to 4° C. and to −16° C. The pH of the blends was measured at room temperature.

### Example 1

### Blends with HMBA and Lysine from ADM

Viscosity, density and pH of Alimet® blends with ADM-Lysine are shown in Table I. The actual Lysine: HMBA ratios were determined by HPLC analysis of each blend for lysine and analysis of HMBA by ion chromatography.

TABLE I
Physical properties of Alimet blends with lysine from ADM

| | Solids % | | | | | |
|---|---|---|---|---|---|---|
| | (wt Lysine + | Viscosity | | Viscosity | | pH at room |
| Lysine:HMBA | HMBA/ | (cSt) | Density at | (cSt) | Density at | temperature |
| ratio | total wt) | at 25° C. | 25 C. (g/mL) | at 4° C. | 4° C. (g/mL) | (22° C.) |
| Lysine | 50% | 41 | 1.123 | 155 | 1.139 | 10.55 |
| 4.56:1 | 54.21% | 53 | 1.128 | 266 | 1.158 | 10.23 |
| 2:1 | 58.52% | 72 | 1.129 | 301 | 1.03 | 9.89 |
| 1.58:1 | 60.06% | 67 | 1.131 | 525 | 1.173 | 9.55 |
| 1:1 | 63.73% | 56 | 1.169 | 637 | 1.246 | 8.4 |
| 1:4 | 76.37% | 60 | 1.171 | 732 | 1.146 | 4.27 |
| Alimet alone | 88% | 76 | 1.12 | 275 | 1.19 | 0.7 |

Viscosity of the Blends

While the viscosity of blends does not change appreciably at 25° C., the viscosity increases as the fraction of Alimet® in the sample increases. The density of the blends also increases with an increase in Alimet® concentration in the blends. At 4° C., the viscosity of lysine, Alimet® and blends thereof are much higher. Also, the viscosity increases with an increase in the concentration of Alimet® in the blend. The increase in viscosity may also be reflective of an increase in the concentration of total solids (lysine+Alimet®) in the sample.

Other Physical Properties

The pH of the blends decreases at the Alimet® concentration increases. It was also found that the various blends shown above did not freeze (solidify) at temperatures of 4° C. as well as −16° C.

### Example 2

Blends with HMBA and Lysine from Ajinomoto (LL-60)

Physical properties of Alimet® blends with lysine from Ajinomoto are shown in Table II. The actual Lysine:HMBA ratios were determined by HPLC analysis of each blend for lysine and analysis of HMBA by ion chromatography.

TABLE II
Physical properties of Alimet blends with lysine from Ajinomoto (LL-60)

| | Solids % | | | | | |
|---|---|---|---|---|---|---|
| | (wt lysine + | Viscosity | | Viscosity | Density | pH at room |
| Lysine:HMBA | HMBA/ | (cSt) | Density at | (cSt) | at 4° C. | temperature |
| ratio | total wt) | at 25° C. | 25° C. (g/mL) | at 4° C. | (g/mL) | (22° C.) |
| Lysine (LL-60) | 60.00% | 266 | 1.16 | 1320 | 1.17 | 10.55 |
| 4:1 | 64% | 371 | 1.2 | 1920 | 1.2 | 10.09 |
| 2:1 | 67.12% | 467 | 1.19 | 4223 | 1.19 | 9.92 |
| 1.46:1 | 67.44% | 529 | 1.19 | 2720 | 1.21 | 9.69 |
| 1:1 | 70.11% | 678 | 1.20 | 7087 | 1.22 | 8.71 |
| 0.3:1 | 79.43% | 348 | 1.22 | 2013 | 1.25 | 3.65 |
| Alimet | 88.00% | 76 | 1.21 | 275 | 1.22 | 0.7 |

Viscosity of the Blends

As can be seen from the table, the viscosity of LL60 is higher than that of ADM-lysine and could be reflective of the higher total lysine concentration in it. At 25° C., the viscosities of blends of LL60 with Alimet are in the range of 400 to 700 cP and at 4° C., it varies between 2000 and 6000 cP. An advantage of LL60 is that it is possible to forms a blend that has a higher solids concentration in it. This is attractive especially from storage and transportation point of view.

Other Physical Properties

The pH of the blends decreases at the Alimet® concentration increases. None of these samples froze at temperatures of 4° C. as well as at −16° C. It should be pointed out at this stage that Alimet forms fairly corrosive mixtures with certain compounds and especially compounds containing chloride ions. However, corrosion rates are usually pH dependent. Consequently, the blends with higher Lysine:Alimet ratios are expected to show acceptable corrosion rates in metal piping systems.

Blends made with Ajinomoto's liquid lysine product having Lysine: HMBA ratios of 2:1 to 1.5:1 appear to have acceptable physical properties near room temperature. However, the high viscosity of the blend at 4° C. may preclude the use of conventional pumps at this temperature for mixing into feed. Systems handling blends of this sort at this temperature may have to be heat traced. An alternate style pump, or alternate delivery systems better suited to handle liquids of high viscosity might be employed to deliver the higher viscosity product. These blends also did not appear to freeze, but became extremely viscous at −16° C.

Blends made with ADM's liquid lysine product having Lys:HMBA ratios of 2:1 to 1.5:1 have acceptable viscosities to permit pumping even at 4° C. Furthermore, the blends appear stable and do not precipitate or freeze at temperatures as low as −16° C.

### Example 3

### HMBA Blends With Reagent Grade Lysine

Having a higher solids (lysine and HMBA) concentration in the HMBA-lysine blends is highly desirable especially from the viewpoint of storage and transportation. Therefore, the properties of high solid blends were studied. Commercially available feed grade lysine has a maximum of 50% lysine free base (ADM lysine) or 60% from Ajinomoto. To make high solids blends, reagent grade lysine free base powder was obtained (Sigma Chemical Co., St. Louis, Mo.), and a lysine solution of total lysine in water prepared. The lysine concentration in this solution was about 66%. This is near the solubility limit of lysine in water. This solution was used to prepare blends of lysine at a lysine:HMBA ratio of 1, 1.5 and 2 and their properties analyzed. Results are summarized in Table III.

TABLE III
Physical properties of Alimet ® blends with reagent grade lysine

| | Solids % | | | | | |
|---|---|---|---|---|---|---|
| | (wt lysine + | | | Viscosity | | pH at room |
| Lysine:HMBA | HMBA/ | Viscosity | Density at | (cP) | Density at | temperature |
| ratio | total wt) | (cP) at 25° C. | 25° C. | at 4° C. | 4° C. (g/mL) | (22° C.) |
| 2:1 | 72.02% | 868 | 1.17 | 4765 | 1.20 | 10.17 |
| 1.5:1 | 73.35% | 1076 | 1.19 | 7030 | 1.22 | 9.00 |
| 1:1 | 76% | 1287 | 1.21 | 7423 | 1.23 | 8.46 |

Viscosity of the Blends

The viscosities of these blends are in the range of 900 to 1300 cst at 25° C. and are between 5000 to 7500 cst at 4° C. At both these temperatures, the viscosity increases with an increase in solids concentration.

All of the references identified hereinabove, are hereby expressly incorporated herein by reference to the extent that they describe, set forth, provide a basis for or enable compositions and/or methods which may be important to the practice of one or more embodiments of the present inventions.

1. An aqueous composition comprising a basic amino acid and 2-hydroxy-4-methylthiobutanoic acid, wherein the mass ratio of basic amino acid to 2-hydroxy-4-methylthiobutanoic acid is between 4:1 and 1:4.

2. The composition of claim 1, wherein said ratio is between 2:1 and 1:1.

3. The composition of claim 1, wherein said basic amino acid is lysine, a lysine salt, or a mixture thereof.

4. The composition of claim 1, wherein the total concentration of lysine and 2-hydroxy-4-methylthiobutanoic acid is less than 77% by weight.

5. The composition of claim 1, wherein the total concentration of lysine and 2-hydroxy-4-methylthiobutanoic acid is less than 65% by weight.

6. A nutritional supplement comprising a blend of a basic amino acid and 2-hydroxy-4-methylthiobutanoic acid, wherein the mass ratio of basic amino acid to 2-hydroxy-4-methylthiobutanoic acid is between 4:1 and 1:4.

7. The nutritional supplement of claim 6, wherein said ratio is between 2:1 and 1:1.

8. The nutritional supplement of claim 6, wherein said basic amino acid is lysine, a lysine salt, or a mixture thereof.

9. The nutritional supplement of claim 6, wherein the total concentration of lysine and 2-hydroxy-4-methylthiobutanoic acid is less than 77% by weight.

10. The nutritional supplement of claim 6, wherein the total concentration of lysine and 2-hydroxy-4-methylthiobutanoic acid is less than 65% by weight.

11. A method of supplementing food, said method comprising: preparing an aqueous mixture of a basic amino acid and 2-hydroxy-4-methylthiobutanoic acid, wherein the mass ratio of basic amino acid to 2-hydroxy-4-methylthiobutanoic acid is between 4:1 and 1:4; and combining said mixture with said food.

12. The method of claim 11, wherein said mass ratio is between 2:1 and 1:1.

13. The method of claim 11, wherein said basic amino acid is lysine, a lysine salt, or a mixture thereof.

14. The method of claim 11, wherein said food is animal feed.

15. The method of claim 11, wherein said food is human food.
